(19) United States
(12) Patent Application Publication
(10) Pub. No.: US 2024/0294995 A1
(43) Pub. Date: Sep. 5, 2024

(54) PRIMER COMBINATION, KIT, AND METHOD FOR DETECTING CLOSTRIDIUM PILIFORME BASED ON LOOP-MEDIATED ISOTHERMAL AMPLIFICATION-LATERAL FLOW DIPSTICK (LAMP-LFD)

(71) Applicant: Hangzhou Medical College, Hangzhou (CN)

(72) Inventors: Fangwei Dai, Hangzhou (CN); Huiqiong Yan, Hangzhou (CN); Wei Han, Hangzhou (CN); Shasang Zhou, Hangzhou (CN); Jiangtao Du, Hangzhou (CN); Junhao Tao, Hangzhou (CN); Honggang Guo, Hangzhou (CN); Huazhong Ying, Hangzhou (CN)

(21) Appl. No.: 18/593,638

(22) Filed: Mar. 1, 2024

(30) Foreign Application Priority Data
Mar. 1, 2023 (CN) 202310216568.6

(51) Int. Cl. C12Q 1/689; C12Q 1/6804; C12Q 1/6806; C12Q 1/6844

(52) U.S. Cl. CPC C12Q 1/689; C12Q 1/6804; C12Q 1/6806; C12Q 1/6844

(57) ABSTRACT

The present disclosure provides a primer combination, a kit, and a method for detecting Clostridium piliforme based on loop-mediated isothermal amplification-lateral flow dipstick (LAMP-LFD), belonging to the technical field of medical molecular biology detection. In the present disclosure, the primer combination includes a forward outer primer F3, a backward outer primer B3, a forward inner primer FIP, a backward inner primer BIP, a loop primer LF, and a probe PB1. The present disclosure further provides a kit and a method for detecting Clostridium piliforme based on the primer combination. The kit and the method can detect the Clostridium piliforme with a simple detection process, a low time consumption, easy determination of results, and high detection accuracy and sensitivity.

### CROSS REFERENCE TO RELATED APPLICATION

This patent application claims the benefit and priority of Chinese Patent Application No. 202310216568.6, filed with the China National Intellectual Property Administration on Mar. 1, 2023, the disclosure of which is incorporated by reference herein in its entirety as part of the present application.

### REFERENCE TO SEQUENCE LISTING

A computer readable XML file entitled “GWP20231109312_sequence listing”, that was created on Mar. 1, 2024, with a file size of about 7,507 bytes, contains the sequence listing for this application, has been filed with this application, and is hereby incorporated by reference in its entirety.

### TECHNICAL FIELD

The present disclosure belongs to the technical field of medical molecular biology detection, and in particular relates to a primer combination, a kit, and a method for detecting Clostridium piliforme based on loop-mediated isothermal amplification-lateral flow dipstick (LAMP-LFD).

### BACKGROUND

Clostridium piliforme, a type of hair-like Clostridium that carries flagella, pili, and spores as well as parasitizes host cells, is widely found in rodents, poultry, livestock, and non-human primates and can cause hemorrhagic necrosis of intestinal tissues and multiple miliary necrosis of the liver in animals. Changes in the external environment or immunosuppression can induce the sudden onset of diseases in animals infected with Clostridium piliforme, leading to the interruption of tumor transplantation experiments. Therefore, Clostridium piliforme is called “the number one disease that destroys cancer research”, such that all countries list the same pathogen that must be eliminated in experimental animals. Clostridium piliforme is a pathogenic bacterium that must be eliminated in clean-level experimental animals during the quality control of experimental animals. Laboratory diagnosis of Clostridium piliforme mainly relies on pathological examination, immunofluorescence assay (IFA), and enzyme-linked immunosorbent assay (ELISA). In China, the national standard GB/T14926.10-2008 for Clostridium piliforme detection methods in experimental animals deletes the cortisone challenge test and only provides serological antibody detection methods. Therefore, it is particularly important to support the biomedical industry of the national economy to establish a rapid, accurate, and convenient method for detecting Clostridium piliforme in experimental animals.

Currently, there are a variety of detection methods for the rapid detection of pathogens, reverse dot blot (RDB), polymerase chain reaction (PCR) and enzyme-linked immuno sorbent assay (ELISA) for rapid immunosorbent assay (ELISA). However, these detection methods have the disadvantages of cumbersome and time-consuming operation, low sensitivity, insufficient specificity, and high cost of instruments and reagents. Loop-mediated isothermal amplification (LAMP) technology was used to design four corresponding LAMP primers for six specific sites on a target gene under a certain temperature and the action of a strand displacement Bst 2.0 DNA polymerase, and then continue to conduct extension and replacement within 1 h. This technology shows simple operation, time saving, high sensitivity and specificity, and low requirements for instruments and equipments. However, LAMP-amplified products are mainly detected using turbidimetry, electrophoresis, and calcein fluorescent dye. During electrophoresis analysis, exposure to the carcinogen EB occurs, and the calcein fluorescent dye is harmful to the human body. These shortcomings reduce the usefulness of LAMP technology in this field. At present, there are no reports on related technologies for detecting Clostridium piliforme based on loop-mediated isothermal amplification-lateral flow dipstick (LAMP-LFD).

### SUMMARY

In view of this, an objective of the present disclosure is to provide a LAMP primer combination for detecting Clostridium piliforme, so as to achieve rapid and accurate detection of the Clostridium piliforme.

The present disclosure further aims to provide a kit for detecting Clostridium piliforme based on LAMP-LFD and a method for detecting Clostridium piliforme based on LAMP-LFD, thus directly determining the presence of Clostridium piliforme through color changes.

To achieve the above objective, the present disclosure provides the following technical solutions:

The present disclosure provides a LAMP primer combination for detecting Clostridium piliforme, including a forward outer primer F3, a backward outer primer B3, a forward inner primer FIP, a backward inner primer BIP, a loop primer LF, and a probe PB1; where
the F3 has a sequence shown in SEQ ID NO: 1, the B3 has a sequence shown in SEQ ID NO: 2, the FIP has a sequence shown in SEQ ID NO: 3, the BIP has a sequence shown in SEQ ID NO: 4, the LF has a sequence shown in SEQ ID NO: 5, and the PB1 has a sequence shown in SEQ ID NO: 6.

Preferably, a 5′-end of the FIP is labeled with biotin, and a 5′-end of the PB1 is labeled with a fluorophore.

Preferably, the fluorophore is selected from the group consisting of carboxyfluorescein (FAM) and fluorescein isothiocyanate (FITC).

The present disclosure further provides use of the primer combination in preparation of a kit for detecting Clostridium piliforme.

The present disclosure provides a kit for detecting Clostridium piliforme based on LAMP-LFD, including the primer combination and an LFD test strip.

Preferably, the outer primer, the inner primer, the loop primer, and the probe are at a concentration ratio of 1:8:4:2.

Preferably, the kit further includes dNTPs, MgSO₄, a Bst2.0 DNA polymerase, a reaction buffer, and ddH₂O.

The present disclosure provides a method for detecting Clostridium piliforme based on LAMP-LFD, where the method is used for a non-diagnostic purpose and includes the following steps: extracting a DNA of a subject to be tested, establishing a LAMP reaction system to allow amplification, and adding a resulting amplified product dropwise to an LFD test strip to allow color development.

Preferably, the LAMP reaction system includes: 2.5 μL of a 10× reaction buffer, 1.25 μL of 100 mM MgSO₄, 1.6 μL of 25 mM dNTPs, 1 μL of 40 μM FIP, 1 μL of 40 μM BIP, 1 μL of 5 μM F3, 1 μL of 5 μM B3, 1 μL of 20 μM LF, 1 μL of 10 μM PB1, 1 μL of an 8 U/μL Bst2.0 DNA polymerase, 1 μL of a DNA template, and 11.65 μL of ddH₂O.

Preferably, the amplification is conducted at 55° C. to 70° C. for 10 min to 60 min.

The present disclosure has following beneficial effects:

In the present disclosure, the LAMP primer combination for detecting Clostridium piliforme is combined with a LAMP amplification principle and LFD to display results using chromatographic test strips, thereby establishing an accurate and specific LAMP-LFD-based detection method for Clostridium piliforme. This method reduces reliance on precision equipment and determines whether the result is negative or positive based on whether a red band appears at the test line. This detection method does not require calcein and hydroxynaphthol blue dyes, thereby eliminating human errors caused by visual identification and observation of color changes. Meanwhile, this method reduces contact with the carcinogen EB when preparing agarose gel electrophoresis, and avoids environmental pollutions and personnel health hazards from radiation, carcinogens EB, and calcein. Accordingly, the present disclosure provides a reliable and effective method for rapid on-site detection of the Clostridium piliforme in experimental animals, and exhibits desirable market application prospects in terms of quality and safety control of the experimental animals.

In the present disclosure, the LAMP-LFD has a minimum concentration of 0.1 fg/μL when detecting the Clostridium piliforme, showing high sensitivity and excellent specificity, and is 100 tim more sensitive than the PCR. Compared with other molecular biology detection methods, the proposed method of the present disclosure is simpler, faster, cheaper, more sensitive, more specific, and more reproducible, and has important market value and prospects.

### BRIEF DESCRIPTION OF THE DRAWINGS

FIG. 1 shows an optimization experiment of a dNTPs concentration of the LAMP reaction system of Clostridium piliforme; where 1 to 6 indicate that the final concentrations are 1.0 mM, 1.2 mM, 1.4 mM, 1.6 mM, 1.8 mM, and 2.0 mM, respectively;

FIG. 2 shows the optimization of the Mg²⁺ content of the LAMP reaction system of Clostridium piliforme where 1 to 7 indicate that the final concentrations were 0 mM, 1 mM, 2 mM, 3 mM, 4 mM, 5 mM, and 6 mM, respectively.

FIGS. 3A-3E show the temperature optimization of the LAMP reaction of Clostridium piliforme where 1 to 5 indicate temperatures of 58° C., 60° C., 62° C., 64° C., and 66° C., respectively.

FIG. 4 shows time optimization of the LAMP-LFD reaction of Clostridium piliforme; where 1 to 8 indicate that the time points are 5 min, 10 min, 15 min, 20 min, 25 min, 30 min, 35 min, and 40 min, respectively;

FIG. 5 shows LAMP-LFD detection results;

FIGS. 6A-6C show LAMP specific detection of Clostridium piliforme; where FIGS. 6A-6B show the repeatability detection of LAMP-LFD; 26: negative control; 1 to 25: Listeria monocytogenes, Salmonella enterica, Shigella dysenteriae, Salmonella typhimurium, Popoff serovar Choleraesuis, S. enterica, Muribacter muris, Salmonella enterica subsp. enterica, Bordetella bronchiseptica, Edwardsiella tarda, Escherichia coli. Corynebacterium kutscheri, Streptococcus suis, Klebsiella oxytoca, Klebsiella pneumoniae, Pseudomonas aeruginosa, Staphylococcus aureus, Aeromonas hydrophila, Salmonella paratyphi A, Pasteurella pneumotropica, Shigella boydii, Shigella flexneri, Klebsiella sp., Salmonella enterica subsp. enterica serovar Pulloru and Clostridium piliforme; FIG. 6C shows the real-time fluorescence quantitative detection of LAMP; 1 to 24: Listeria monocytogenes, Salmonella enterica, Shigella dysenteriae, Salmonella typhimurium, Popoff serovar Choleraesuis, S. enterica, Muribacter muris, Salmonella enterica subsp. enterica, Bordetella bronchiseptica, Edwardsiella tarda, Escherichia coli. Corynebacterium kutscheri, Streptococcus suis, Klebsiella oxytoca, Klebsiella pneumoniae, Pseudomonas aeruginosa, Staphylococcus aureus, Aeromonas hydrophila, Salmonella paratyphi A, Pasteurella pneumotropica, Shigella boydii, Shigella flexneri, Klebsiella sp., Salmonella enterica subsp. enterica serovar Pullorum; +: positive control;

FIGS. 7A-7B show LAMP sensitivity detection of Clostridium piliforme; where FIG. 7A represents the LAMP-LFD sensitivity detection; FIG. 7B represents the PCR sensitivity detection; N: negative control; 1 to 9 indicates that the concentrations of the Clostridium piliforme are 1 ng/μL, 0.1 ng/μL, 0.01 ng/μL, 1 pg/μL, 0.1 pg/μL, 0.01 pg/μL, 1 fg/μL, 0.15 fg/μL, and 0.01 fg/μL;

FIG. 8 shows the repeatability of LAMP-LFD detection of Clostridium piliforme, where 2/4/6 are negative controls, while 1/3/5 are groups of Clostridium piliforme with a concentration of 0.1 fg/μL.

FIGS. 9A-9B show LAMP-LFD detection of Clostridium piliforme in clinical samples; FIG. 9A: detection of liver DNA samples; 1 to 8: liver DNA samples; N: negative control; P: positive control; FIG. 9B: detection of fecal DNA samples; 1 to 4: fecal DNA samples; N: negative control; P: positive control.

### DETAILED DESCRIPTION OF THE EMBODIMENTS

The present disclosure targets a 23S rRNA gene of the Clostridium piliforme and provides a LAMP primer combination for detecting Clostridium piliforme, including a forward outer primer F3, a backward outer primer B3, a forward inner primer FIP, a backward inner primer BIP, a loop primer LF, and a probe PB1; where a 196 bp target fragment can be amplified.

In the present disclosure, F3 has the following sequence: 5′-GCTCTGCTACTGTATACTGAA-3′ shown in SEQ ID NO: 1; B3 has a sequence: 5′-ACAATTCGACTATCTCTCATCA-3, ‘ as shown in SEQ ID NO: 2; FIP has a sequence: 5’-CCGCTACTTAGGAAATCGATTTTTCGGGGAACGTTGTGAACTG-3′ shown in SEQ ID NO: 3; BIP has the following sequence: 5′-CGAGCGAAAGGGAAAGAGGC-ATGGATTTTGCAGTCCTCAA-3′ shown in SEQ ID NO: 4; LF has a sequence: 5′-CTCTTCCTGTTGCTACTTAGATGTT-3′ shown in SEQ ID NO: 5; PB1 has a sequence: 5′-GCCAAACCATAAAGCGTGC-3′ shown in SEQ ID NO: 6.

In the present disclosure, the 5′-end of the FIP was labeled with biotin, and the 5′-end of PB1 was labeled with a fluorophore; the fluorophore included FAM or FITC, preferably FAM.

The present disclosure further demonstrates the use of a primer combination in the preparation of a kit for detecting Clostridium piliforme.

The present disclosure provides a kit for detecting Clostridium piliforme based on LAMP-LFD, including the primer combination and an LFD test strip. Preferably, the outer primer, inner primer, loop primer, and probe in the primer combination had a concentration ratio of 1:8:4:2.

In the present disclosure, the kit further included dNTPs, MgSO₄, a Bst 2.0 DNA polymerase, a reaction buffer, and ddH₂O. The reaction buffer is preferably a 10× reaction buffer; the dNTPs have a concentration of 1.0 mM to 2.0 mM, preferably 1.6 mM; Mg²⁺ in the reaction system has a final concentration of 4 mM to 10 mM (1 μL to 2.5 μL), preferably 5 mM.

The present disclosure provides a method for detecting Clostridium piliforme based on LAMP-LFD, which is used for non-diagnostic purposes and includes the following steps: extracting the DNA of a subject to be tested, establishing a LAMP reaction system to allow amplification, and adding a resulting amplified product dropwise to an LFD test strip to allow color development. In the present disclosure, amplification and hybridization were conducted simultaneously. The DNA of the subject to be tested is the DNA template in the LAMP reaction system; the DNA template includes but is not limited to DNA samples obtained from pure culture colonies and plasmids; when the subject to be tested is rodents, poultry, livestock, and non-human primates, preferably fecal or visceral DNA is extracted as the DNA template in the LAMP reaction system.

In the present disclosure, the LAMP reaction system includes: 2.5 μL of a 10× reaction buffer, 1.25 μL of 100 mM MgSO₄, 1.6 μL of 25 mM dNTPs, 1 μL of 40 μM FIP, 1 μL of 40 μM BIP, 1 μL of 5 μM F3, 1 μL of 5 μM B3, 1 μL of 20 μM LF, 1 μL of 10 μM PB1, 1 μL of an 8 U/μL Bst 2.0 DNA polymerase, 1 μL of a DNA template, and 11.65 μL of ddH₂O.

In the present disclosure, the LAMP reaction system was reacted at 55-70° C., preferably 58-66° C., preferably 60-64° C. for 10-60 min, preferably 15-40 min, and 15-30 min.

In the present disclosure, the color development preferably includes mixing the LAMP reaction product and ddH₂O, dropwise adding 40 μL to 60 μL of a resulting mixed system to a sample addition zone of the LFD test strip, where the obtained reaction product and ddH₂O are at a volume ratio of 1:(20-100), preferably 1:50; and the mixed system is preferably 50 μL.

In the present disclosure, the color development specifically includes placing the LFD test strip horizontally for 5-10 min and then observing the results, adding a resulting diluted reaction product dropwise into the sample addition zone of the LFD test strip, migrating forward through a capillary action, and passing through a sample conjugate pad, a test line, and a control line in sequence. If a target gene is present in the reaction system, a biotin-FAM-labeled DNA probe in the reaction product is combined with a colloidal gold-labeled FAM antibody on the conjugate pad to form a ternary complex. At this time, the ternary complex was captured by a biotinylated antibody on the test line and appeared as a red band on the test and control lines. If there is no target gene in the reaction system, the FAM-labeled probe can directly bind to the colloidal gold-labelled FAM antibody on the conjugate pad to form a binary complex without biotin participation, which will only show a red band on the control line.

In the present disclosure, the detection method of Clostridium piliforme is simple and convenient to operate and does not require large precision instruments. The entire detection process relies only on a water bath and test strips, and takes approximately 45 mins to complete.

The technical solution provided by the present disclosure is described in detail below with reference to the examples, but they should not be construed as limiting the scope of the present disclosure.

In the following examples, a bacterial DNA extraction kit was purchased from TIANGEN Biotech (Beijing) Co., Ltd. a PCR amplification kit was purchased from Takara Bio Co., Ltd. Bst 2.0 DNA polymerase was purchased from NEB Biolabs Co., Ltd. and a dNTPs mixture was purchased from Sangon Biotech (Shanghai) Co., Ltd.

In the following examples, all methods are conventional methods, unless otherwise specified.

All materials and reagents used in the following examples may be commercially available, unless otherwise specified.

### Example 1

### 1. Bacterial Culture and DNA Extraction

The Clostridium piliforme antigen pieces stored in the laboratory were scraped with a sterile cotton swab in a biological safety cabinet and transferred to a 1.5 mL centrifuge tube filled with ddH₂O. Clostridium piliforme DNA was extracted according to the bacterial DNA extraction instructions, and the concentration and purity of the DNA sample were determined by measuring the absorbance at A260 and A280 nm using a NanoDrop UV spectrophotometer. The sample was stored in −20° C. refrigerator for later use.

### 2. LAMP Primer and Probe Design

After logging into the NCBI website and entering GenBank, the 23SrRNA gene sequence of Clostridium piliforme was searched and downloaded, with a GenBank sequence number DQ352811.1. Multiple sets of LAMP primers and probes were designed for 23SrRNA using Primer Explorer V5 software, and the forward outer primer F3, backward outer primer B3, forward inner primer FIP, backward inner primer BIP, loop primer LF, and probe PB1 were finally selected: F3 had a sequence shown in SEQ ID NO: 1, B3 had a sequence shown in SEQ ID NO: 2, FIP had a sequence shown in SEQ ID NO: 3, BIP had a sequence shown in SEQ ID NO: 4, LF had a sequence shown in SEQ ID NO: 5, and PB1 sequence was shown in SEQ ID NO: 6.

All the above primers and probes were synthesized and labeled by Shanghai Sangon.

3. Construction of Plasmid of Clostridium piliforme

The external primers F3 and B3 were used as PCR reaction primers, and the target bands were amplified using the PCR reaction kit. The amplified product was subjected to agarose gel electrophoresis. The gel was cut under UV light and the product was recovered using an agarose gel recovery kit. The recovered products were ligated and transformed according to the instructions of the pMD18-Tvector. Transformed competent cells were evenly spread on Amp LB agar medium and cultured overnight. A single colony was selected on the culture medium, colony PCR was performed, and the positive target product was sent to Beijing Qingke Biotechnology Co., Ltd. for sequencing. Colonies with the correct sequence comparisons were frozen and preserved.

### 4. Establishment of LAMP Reaction System

The extracted Clostridium piliforme DNA was used as a template, and the entire process was completed in a 25 μL reaction system. The reaction system included 2.5 μL 10× reaction buffer, 1.5 μL MgSO₄ (100 mM), 1.6 μL dNTPs (25 mM), 1 μL 40 μM FIP, 1 μL 40 μM BIP, 1 μL 5 μM F3, 1 μL 5 μM B3, 1 μL Bst 2.0 DNA polymerase (8 U/μL), 1 μL LF, 0.5 μL LAMP fluorescent dye, 1 μL DNA template, and 11.9 μL ddH₂O.

Sterile ddH₂O was used as a negative control. The above solutions were sequentially added to a 100 μL reaction tube. Fifty microliters of mineral oil was added to seal the sample, briefly centrifuged, and verified using a Bio-Rad CFX-96 Touch real-time fluorescence quantitative PCR instrument. The amplification program included: 1 min×60 at 62° C. and 10 min at 85° C.

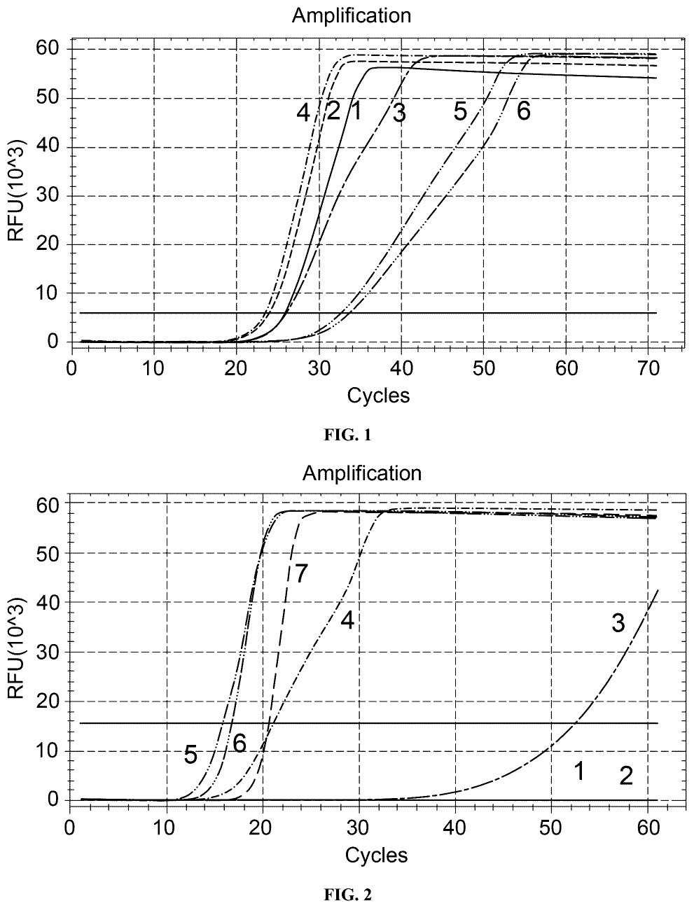

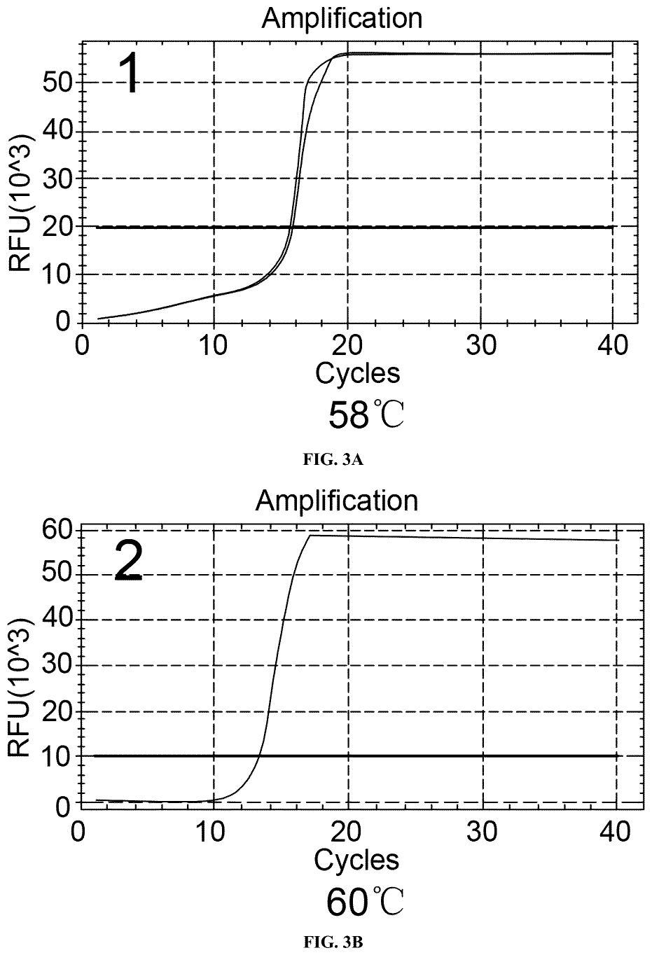

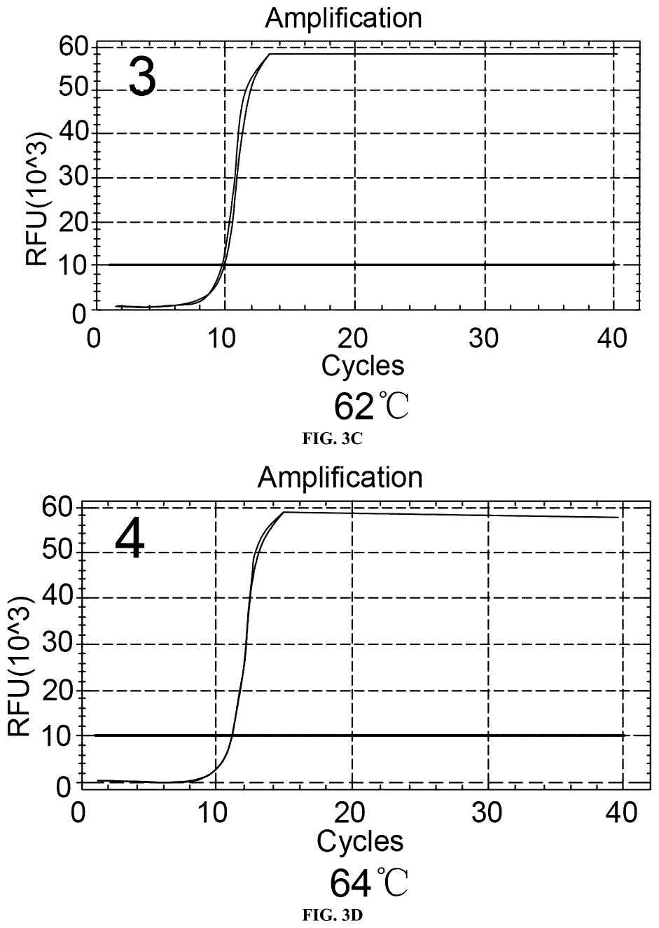

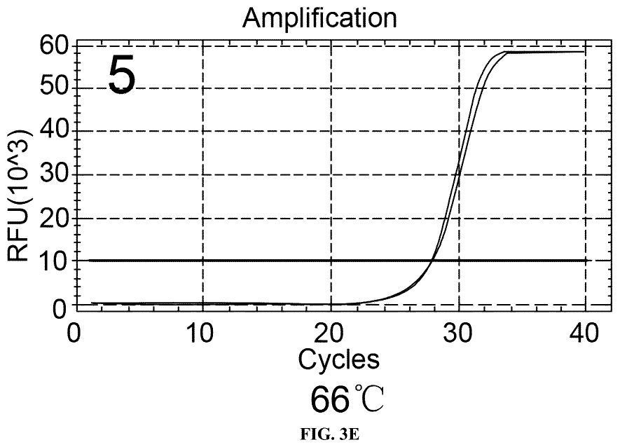

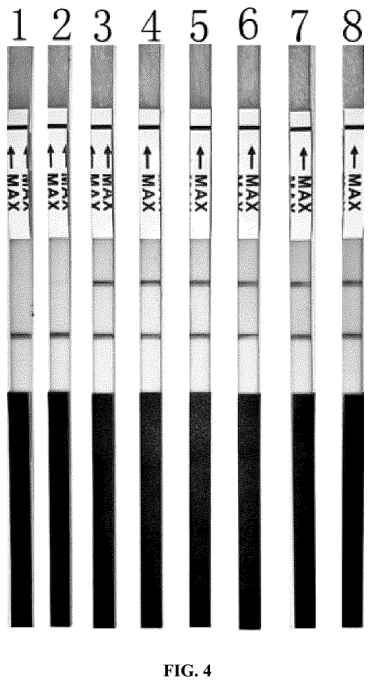

The reaction conditions and parameters were optimized based on the above reaction system.
(1) The concentration of dNTPs in the reaction system was optimized, the final concentration of dNTPs in the reaction system was selected to be 1.0 mM to 2.0 mM, the concentration was increased by 0.2 mM, and the detection was repeated 3 times. The results for different concentrations of dNTPs in the reaction system are shown in FIG. 1. As shown in FIG. 1, the amplification curve was optimal when the final concentration of dNTPs was 1.6 mM, such that the optimal final concentration of dNTPs in the reaction system was 1.6 mM.
(2) The best amplification curve for this reaction was selected. When the final concentration of dNTPs was 1.6 mM, the Mg²⁺ content was optimized, and the Mg²⁺ content was subjected to LAMP at intervals of 0.25 μL from 0 μL-1.5 μL (at a final concentration of 0-6 mM). The optimized results are shown in FIG. 2. When the reaction system did not contain Mg²⁺ or the Mg²⁺ content was 0.25 μL (at a final concentration of 1 mM), the LAMP efficiency was zero and no amplification curve appeared. The amplification curve appeared when the Mg²⁺ content was in the range of 0.5 μL to 1.5 μL (final concentration 2 mM to 6 mM), and the amplification curve first appeared at 1.25 μL (final concentration 5 mM), showing the best amplification effect. The most suitable amount of Mg²⁺ in this reaction system was 1.25 μL (final concentration, 5 mM).
(3) The reaction temperature was optimized based on the optimal reaction system described above. The set temperatures were 58, 60, 62, 64, and 66° C. The reaction temperature affected the amplification efficiency. The optimization results are shown in FIGS. 3A-3E. Amplification curves appeared in the temperature range of 58-66° C.; and when the temperature was 62° C., the amplification curve appeared first, and 62° C. was finally selected as the optimal reaction temperature.
(4) The reaction time was optimized according to the optimized reaction conditions, and the time range was set to 5 min to 40 min; and 5 min was set as the time interval to detect by LFD, and the reaction time was selected to correspond to the most obvious band as the best reaction time. There was a relationship between reaction time and primer binding ability. After three parallel experiments, the optimized results are shown in FIG. 4. When the reaction time was 5 min, no target band appeared; the target band was obviously produced within 15 min of the reaction time, and reached its brightest level. Thus, 15 min was selected as the optimal reaction time.

A series of optimization of reaction parameters was conducted through single-factor optimization experiments, and the optimal LAMP reaction system of 25 μL was finally determined to include: 2.5 μL 10× reaction buffer, 1.25 μL MgSO₄ (100 mM), 1.6 μL dNTPs (25 mM), 1 μL 40 μM FIP, 1 μL 40 μM BIP, 1 μL 20 μM LF, 1 μL 5 μM F3, 1 μL 5 μM B3, 1 μL Bst 2.0 DNA polymerase (8 U/μL), 1 μL DNA template, and 12.65 μL ddH₂O. The reaction was conducted in a Bio-Rad CFX-96 Touch real-time fluorescence quantitative PCR instrument. The amplification program included 62° C., 1 min×60, and continued heating at 85° C. for 10 min to inactivate the enzyme.

### Example 2

This example provided the LAMP-LFD experimental method:

The primer sequence was the same as that in Example 1, where the 5′-end of FIP was labeled with biotin (5′-Biotin) and the 5′-end of PB1 was labeled with FAM (5′-6-FAM).

Under the optimal LAMP reaction conditions in Example 1, LAMP was conducted using Clostridium piliforme DNA as a template. In the reaction system, biotin-labeled FIP was used to replace FIP, and 1 μL of FAM-labeled probe PB1 at a concentration of 20 pmol was added to the reaction system, while reducing the amount of ddH₂O. The reaction was conducted in a water bath at 62° C. for 60 min and 85° C. for 10 min.

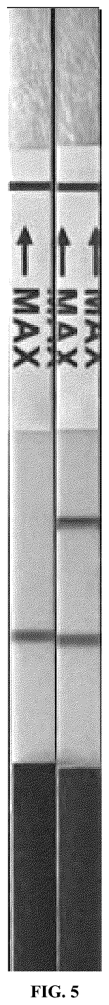

The reaction product (2 μL) was added to 100 μL of Sample diluent, and 50 μL of the diluted reaction product was added dropwise into a sample addition zone of the test strip. The results were observed after allowing it stand horizontally for 5 min. As shown in FIG. 5, red bands appeared on both the test line and the control line for positive results, and red bands appeared only on the control line for negative results, indicating that LAMP-LFD was successfully established.

### Example 3

### 1. Analysis of Specificity for LAMP-LFD Experimental Method

The DNA samples extracted from Clostridium piliforme, Listeria monocytogenes, Salmonella enterica, Shigella dysenteriae, Salmonella typhimurium, Popoff serovar Choleraesuis, S. enterica, Muribacter muris, Salmonella enterica subsp. enterica, Bordetella bronchiseptica, Edwardsiella tarda, Escherichia coli. Corynebacterium kutscheri, Streptococcus suis, Klebsiella oxytoca, Klebsiella pneumoniae, Pseudomonas aeruginosa, Staphylococcus aureus, Aeromonas hydrophila, Salmonella paratyphi A, Pasteurella pneumotropica, Shigella boydii, Shigella flexneri, Klebsiella sp., Salmonella enterica subsp. enterica serovar Pullorum and Clostridium piliforme were used as template, sterile ddH₂O was used as a negative control, the Clostridium piliforme DNA was used as a positive control, and the LAMP-LFD experimental method in Example 2 was used for detection.

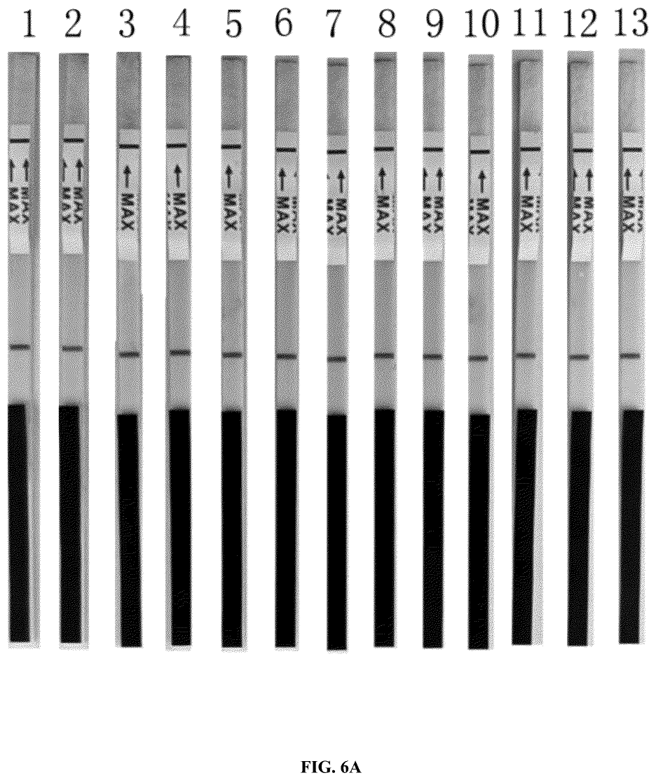

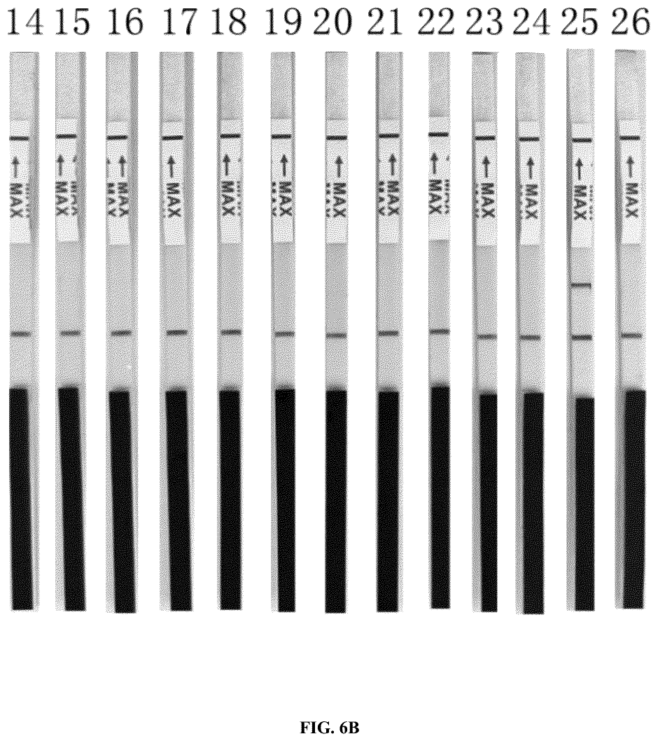

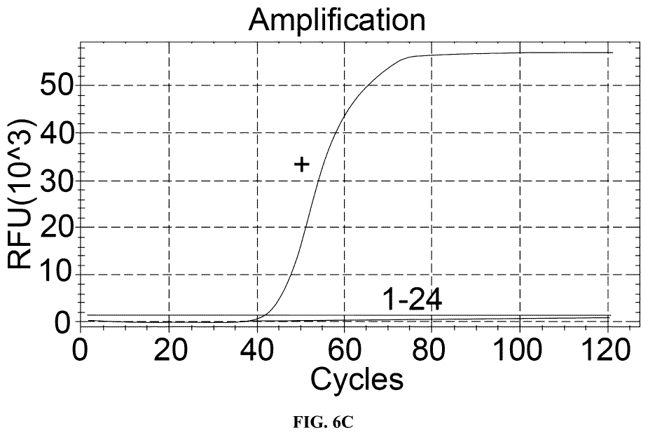

The tests results are shown in FIGS. 6A-6C. When Clostridium piliforme genome was used as a template, two obvious red bands appeared on the test line and control line of the lateral chromatography test strip, indicating that the test result was positive. When non-Clostridium piliforme and sterile ddH₂O were used as templates, no red band appeared at the test line of the lateral chromatography test strip, indicating a negative test result. This indicated that the established detection method had a desirable specificity.

### 2. Analysis of Sensitivity for LAMP-LFD Experimental Method

The genomic DNA of the Clostridium piliforme was diluted with sterile ddH₂O according to a 10-fold gradient, and the final DNA concentration ranged from 25 ng/μL to 0.25 fg/μL. DNA at different concentrations was used as a template, and the LAMP-LFD experimental method described in Example 2 was used for detection.

Simultaneously, PCR was performed conducted as a control. The reaction system included: 12.5 μL of 2× Estaq Master Mix (Dye), 2 μL each of F3 and R3 primers, 1 μL of template DNA, and adding ddH₂O to make up to 25 μL (the concentration range of DNA in 25 μL of the reaction system was 1 ng/μL to 0.01 fg/μL). After 30 cycles of initial denaturation at 94° C. for 3 min, denaturation at 94° C. for 30 s, annealing at 56° C. for 30 s, and extension at 72° C. for 60 s, and a final extension at 72° C. for 5 min. PCR products were subjected to 1.5% agarose gel electrophoresis for detection.

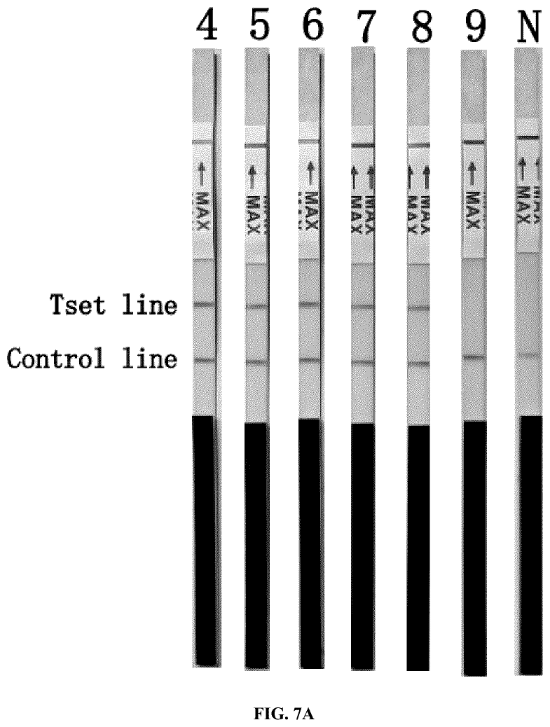

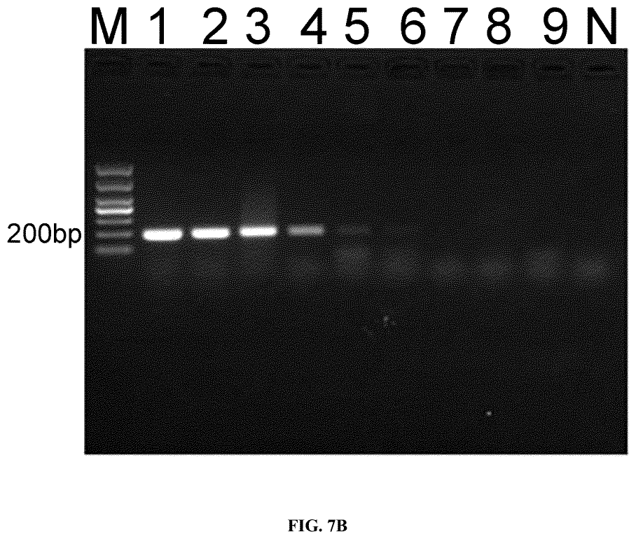

The results were shown in FIGS. 7A-7B: as shown in FIG. 7A, the LAMP-LFD experiment was conducted using DNA in the concentration range of 25 pg/μL to 0.25 fg/μL as the template (the concentration range of DNA in 25 μL of the reaction system was in the range of 1 pg/μL to 0.01 fg/μL), the minimum detection concentration of LAMP-LFD experimental method was 0.1 fg/μL. As shown in FIG. 7B, the PCR experiment was conducted using DNA in the concentration range of 25 ng/μL to 0.25 fg/μL as the template (the concentration range of DNA in PCR of the 25 μL reaction system was in the range of 1 ng/μL to 0.01 fg/μL), the minimum detection concentration PCR experimental method was 0.01 pg/μL. The lowest DNA concentration detected by LAMP-LFD in a sample was 100 times that of conventional PCR detection methods with high sensitivity.

### 3. LAMP-LFD Repeated Experiments

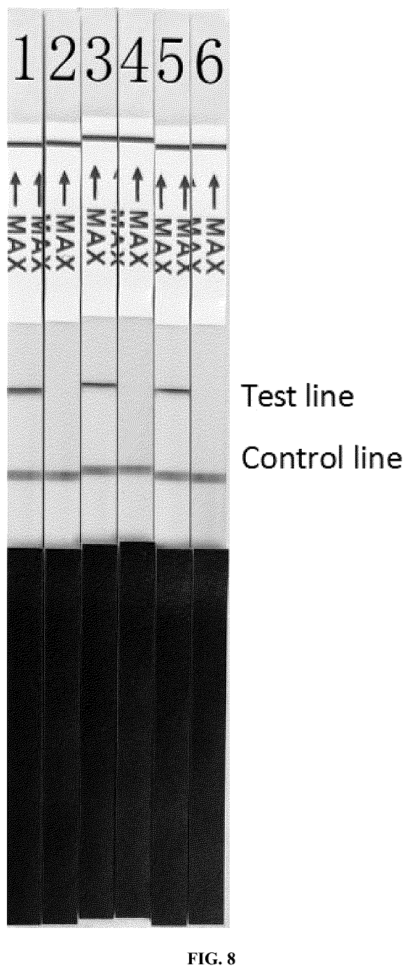

Three sets of parallel samples were prepared to determine method reproducibility, the Clostridium piliforme DNA with a known concentration was diluted to 2.5 fg/μL with ddH₂O, and then used as a template to allow detection using the LAMP-LFD experimental method in Example 2 (the concentration of DNA in 25 μL of the reaction system was 0.1 fg/μL). The results are shown in FIG. 8; and the same results were obtained in three parallel reactions, indicating that the established LAMP-LFD for detecting Clostridium piliforme had desirable repeatability, and the lowest detection concentration was stabilized at 0.1 fg/μL.

### Example 4

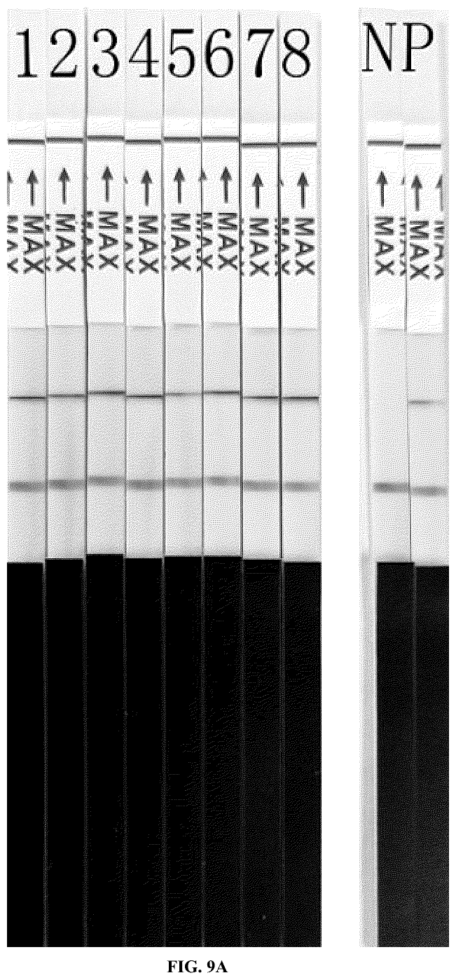

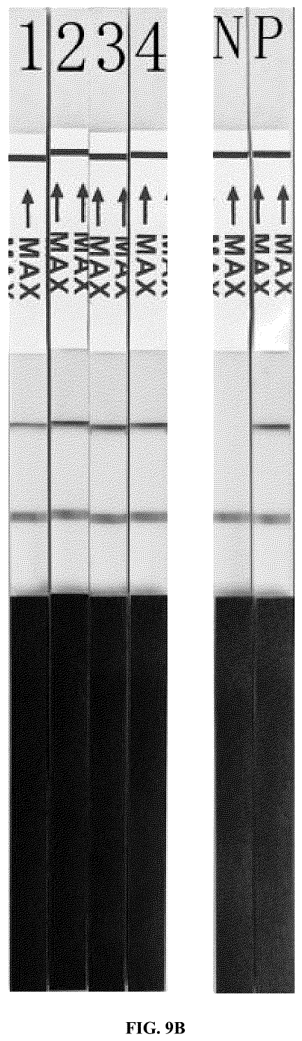

To determine the feasibility of LAMP-LFD detection in practical applications, DNA was extracted from the liver and feces of gerbils infected with Clostridium piliforme and detected using the LAMP-LFD method in Example 2. The results were shown in FIGS. 9A-9B: in the DNA samples extracted from the liver and feces, the test line and control line of the lateral chromatography test strip showed two obvious red bands. This showed that the established LAMP-LFD method for detecting Clostridium piliforme was feasibility for practical application.

The above descriptions are merely the preferred implementation of the present disclosure. It should be noted that a person with ordinary skill in art may further make several improvements and modifications without departing from the principle of the present disclosure, but such improvements and modifications should be deemed as falling within the protection scope of the present disclosure.

What is claimed is:

1. A loop-mediated isothermal amplification (LAMP) primer combination for detecting Clostridium piliforme, comprising a forward outer primer F3, a backward outer primer B3, a forward inner primer FIP, a backward inner primer BIP, a loop primer LF, and a probe PB1; wherein
the F3 has a sequence shown in SEQ ID NO: 1, the B3 has a sequence shown in SEQ ID NO: 2, the FIP has a sequence shown in SEQ ID NO: 3, the BIP has a sequence shown in SEQ ID NO: 4, the LF has a sequence shown in SEQ ID NO: 5, and the PB1 has a sequence shown in SEQ ID NO: 6.

2. The primer combination according to claim 1, wherein a 5′-end of the FIP is labeled with biotin, and a 5′-end of the PB1 is labeled with a fluorophore.

3. The primer combination according to claim 2, wherein the fluorophore is selected from the group consisting of carboxyfluorescein (FAM) and fluorescein isothiocyanate (FITC).

4. A method for preparation of a kit for detecting Clostridium piliforme using the primer combination according to claim 1.

5. A method for preparation of a kit for detecting Clostridium piliforme using the primer combination according to claim 2.

6. A method for preparation of a kit for detecting Clostridium piliforme using the primer combination according to claim 3.

7. A kit for detecting Clostridium piliforme based on loop-mediated isothermal amplification-lateral flow dipstick (LAMP-LFD), comprising the primer combination according to claim 1, and an LFD test strip.

8. A kit for detecting Clostridium piliforme based on loop-mediated isothermal amplification-lateral flow dipstick (LAMP-LFD), comprising the primer combination according to claim 2, and an LFD test strip.

9. A kit for detecting Clostridium piliforme based on loop-mediated isothermal amplification-lateral flow dipstick (LAMP-LFD), comprising the primer combination according to claim 3, and an LFD test strip.

10. The kit according to claim 7, wherein the outer primer, the inner primer, the loop primer, and the probe are at a concentration ratio of 1:8:4:2.

11. The kit according to claim 8, wherein the outer primer, the inner primer, the loop primer, and the probe are at a concentration ratio of 1:8:4:2.

12. The kit according to claim 9, wherein the outer primer, the inner primer, the loop primer, and the probe are at a concentration ratio of 1:8:4:2.

13. The kit according to claim 7, further comprising dNTPs, MgSO₄, a Bst2.0 DNA polymerase, a reaction buffer, and ddH₂O.

14. The kit according to claim 8, further comprising dNTPs, MgSO₄, a Bst2.0 DNA polymerase, a reaction buffer, and ddH₂O.

15. The kit according to claim 9, further comprising dNTPs, MgSO₄, a Bst2.0 DNA polymerase, a reaction buffer, and ddH₂O.

16. A method for detecting Clostridium piliforme based on LAMP-LFD, wherein the method is used for a non-diagnostic purpose and comprises the following steps: extracting a DNA of a subject to be tested, establishing a LAMP reaction system to allow amplification, and adding a resulting amplified product dropwise to an LFD test strip to allow color development.

17. The method according to claim 16, wherein the LAMP reaction system comprises: 2.5 μL of a 10× reaction buffer, 1.25 μL of 100 mM MgSO₄, 1.6 μL of 25 mM dNTPs, 1 μL of 40 μM FIP, 1 μL of 40 μM BIP, 1 μL of 5 μM F3, 1 μL of 5 μM B3, 1 μL of 20 μM LF, 1 μL of 10 μM PB1, 1 μL of an 8 U/μL Bst 2.0 DNA polymerase, 1 μL of a DNA template, and 11.65 μL of ddH₂O.

18. The method according to claim 16, wherein the amplification is conducted at 55° C. to 70° C. for 10 min to 60 min.


SEQUENCE LISTING (6 sequences)

SEQ ID NO: 1 - DNA, 21 bp, synthetic construct
  source 1..21: mol_type = other DNA; note = Sequence of forward outer primer F3
gctctgctactgtatactgaa

SEQ ID NO: 2 - DNA, 22 bp, synthetic construct
  source 1..22: mol_type = other DNA; note = Sequence of reverse outer primer B3
acaattcgactatctctcatca

SEQ ID NO: 3 - DNA, 43 bp, synthetic construct
  source 1..43: mol_type = other DNA; note = Sequence of forward inner primer FIP
ccgctacttaggaaatcgatttttcggggaacgttgtgaactg

SEQ ID NO: 4 - DNA, 40 bp, synthetic construct
  source 1..40: mol_type = other DNA; note = Sequence of reverse inner primer BIP
cgagcgaaagggaaagaggcatggattttgcagtcctcaa

SEQ ID NO: 5 - DNA, 25 bp, synthetic construct
  source 1..25: mol_type = other DNA; note = Sequence of loop primer LF
ctcttcctgttgctacttagatgtt

SEQ ID NO: 6 - DNA, 19 bp, synthetic construct
  source 1..19: mol_type = other DNA; note = Sequence of probe PB1
gccaaaccataaagcgtgc